(19) United States
(12) Patent Application Publication
(10) Pub. No.: US 2013/0037788 A1
(43) Pub. Date: Feb. 14, 2013

(54) NOVEL m-TERPHENYL COMPOUND AND ORGANIC LIGHT EMITTING DEVICE INCLUDING THE SAME

(71) Applicants: Tetsuya Kosuge, Yokohama-shi (JP); Jun Kamatani, Tokyo (JP); Kengo Kishino, Tokyo (JP); Hiroyuki Tomono, Numazu-shi (JP)

(72) Inventors: Tetsuya Kosuge, Yokohama-shi (JP); Jun Kamatani, Tokyo (JP); Kengo Kishino, Tokyo (JP); Hiroyuki Tomono, Numazu-shi (JP)

(73) Assignee: CANON KABUSHIKI KAISHA, Tokyo (JP)

(21) Appl. No.: 13/642,477

(22) PCT Filed: Apr. 11, 2011
(86) PCT No.: PCT/JP2011/059467

§ 371 Date: Oct. 19, 2012

(30) Foreign Application Priority Data
Apr. 22, 2010 (JP) 2010-099153

(51) Int. Cl. H01L 51/54; C07C 15/30; C07C 15/38; C07D 333/76

(52) U.S. Cl. 257/40; 549/43; 585/26; 257/E51.026

(57) ABSTRACT

An organic light emitting device which includes a m-terphenyl compound having a high T1 energy is provided. In addition, a novel m-terphenyl compound is provided.

### TECHNICAL FIELD

The present invention relates to a novel m-terphenyl compound and an organic light emitting device including the same.

### BACKGROUND ART

An organic light emitting device is a device including an anode, a cathode, and an organic compound layer disposed between these two electrodes. In the organic light emitting device, excitons are generated when holes and electrons, which are injected from the respective electrodes, are recombined within the organic compound layer, and light is emitted when the excitons return to the ground state. Recent advancements of the organic light emitting device have been remarkable, and a thin and light-weight light emitting device having a low driving voltage, various light emitting wavelengths, and a high speed response can be formed.

A phosphorescence light emitting device is an organic light emitting device which includes a phosphorescence material in the organic compound layer and which can emit light derived from triplet excitons of the above phosphorescence material. However, the light emitting efficiency of the phosphorescence light emitting device can still be improved.

As a material used for a light emitting layer of the phosphorescence light emitting device, for example, the following compounds H01 and H02 have been disclosed in PTLs 1 and 2, respectively.

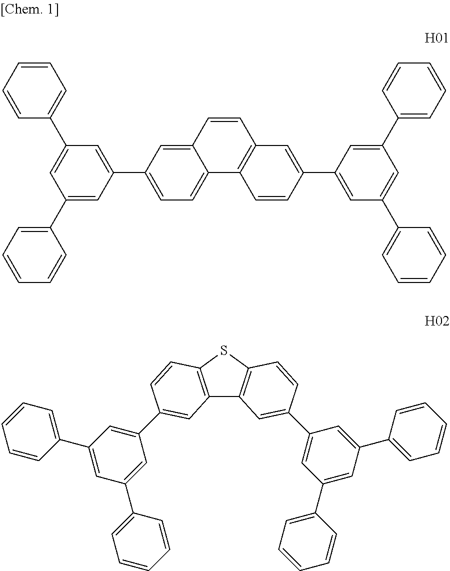

### CITATION LIST

### Patent Literature

PTL 1 Japanese Patent Laid-Open No. 2009-215333
PTL 2 International Publication No. WO2007/111176 pamphlet

### SUMMARY OF INVENTION

### Technical Problem

It has been believed that the compounds disclosed in PTLs 1 and 2 can still be improved.

The compound H01 having a phenanthrene ring can still be improved in order to increase the lowest excited triplet state energy (T1 energy) and to deepen the LUMO level (to increase the electron affinity). In addition, the compound H02 having a dibenzothiophene ring can also still be improved in order to increase the T1 energy and to deepen the LUMO level.

The present invention provides a novel m-terphenyl compound having a high T1 energy and a deep LUMO level. Furthermore, by using the novel m-terphenyl compound described above, the present invention provides an excellent organic light emitting device having a high light emitting efficiency and a low driving voltage.

Accordingly, the present invention provides a m-terphenyl compound represented by the following general formula [1].

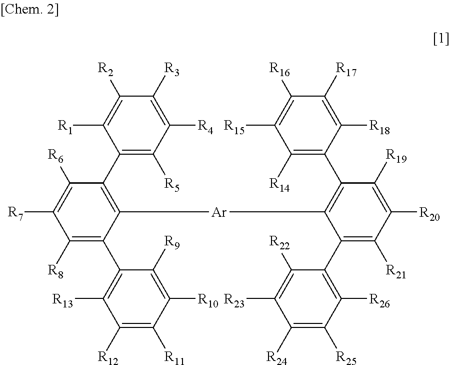

In formula [1], R1 to R26 each independently indicate a hydrogen atom or an alkyl group having 1 to 4 carbon atoms. Ar is selected from arylene groups shown in formula [2].

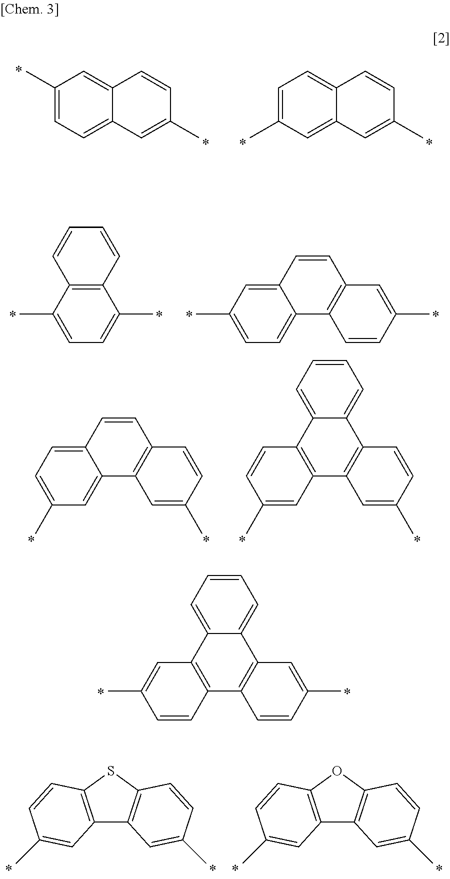

In formula [2], * indicates a bonding site to the m-terphenyl group.

### BRIEF DESCRIPTION OF DRAWING

FIG. 1 is a schematic cross-sectional view showing an organic light emitting device and a switching element connected thereto.

### DESCRIPTION OF EMBODIMENT

A m-terphenyl compound according to the present invention will be described.

The m-terphenyl compound according to the present invention is represented by the following general formula [1].

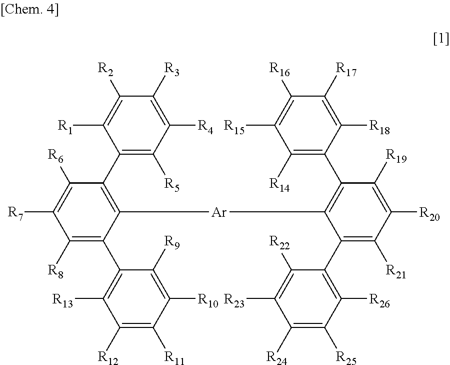

In formula [1], R₁ to R₂₆ each independently indicate a hydrogen atom or an alkyl group having 1 to 4 carbon atoms.

As particular examples of the alkyl group having 1 to 4 carbon atoms, there are a methyl group, an ethyl group, an n-propyl group, an iso-propyl group, an n-butyl group, an iso-butyl group, a sec-butyl group, and a tert-butyl group.

The above alkyl groups each may have a substituent. For example, there may be mentioned a hydrocarbon aromatic ring group, such as a phenyl group, a naphthyl group, a phenanthryl group, or a fluorenyl group; a heteroaromatic ring group, such as a thienyl group, a pyrrolyl group, or a pyridyl group; a substituted amino group, such as a dimethylamino group, a diethylamino group, a dibenzylamino group, a diphenylamino group, a ditolylamino group, or a dianisolylamino group; an alkoxy group, such as a methoxy group or an ethoxy group; an aryloxy group, such as a phenoxy group or a naphthoxy group; a halogen atom, such as fluorine, chlorine, bromine, or iodine; a hydroxyl group; a cyano group; or a nitro group.

Ar of formula [1] is selected from arylene groups shown in formula [2].

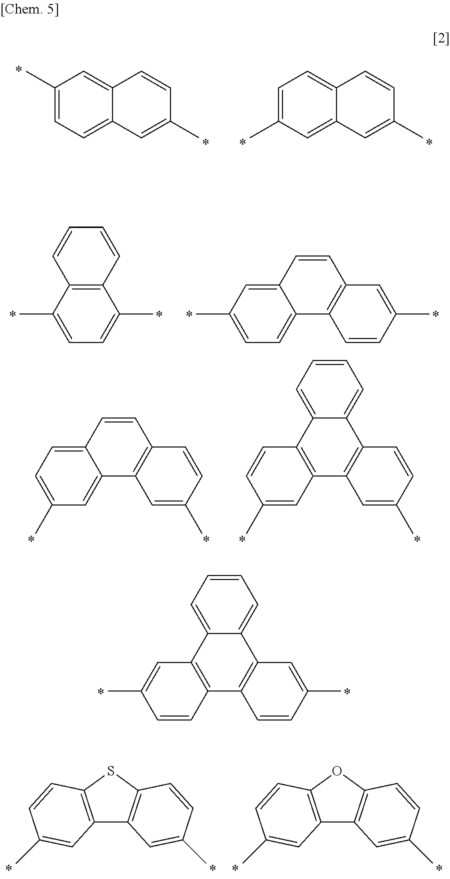

In formula [2], * indicates a bonding site to the m-terphenyl group.

A partial structure represented by Ar of formula [1], that is, a naphthalene ring, a phenanthrene ring, a triphenylene ring, a dibenzothiophene ring, and a dibenzofuran ring shown in formula [2], will be described as a central condensed ring of the compound according to the present invention.

Properties of m-Terphenyl Compound According to Present Invention

The T1 energy (equivalent wavelength) and the LUMO level (calculated value) of each of main simple central condensed rings are shown in the following Table 1. Condensed rings having a T1 energy of 480 nm or less and a deep LUMO level of −0.9 eV or less are the condensed rings shown by formula [2], each of which can be used as the central condensed ring of the compound according to the present invention.

TABLE 1

| | | T1 ENERGY | LUMO LEVEL |
|---|---|---|---|
| | STRUCTURAL | EQUIVALENT | CALCULATED |
| | FORMULA | WAVELENGTH | VALUE |
| NAPHTHALENE | 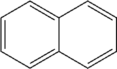 | 472 nm | −0.96 eV |
| PHENANTHRENE | 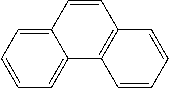 [SMILES: c1ccc2c(c1)ccc1ccccc12] | 459 nm | −0.99 eV |
| TRIPHENYLENE | 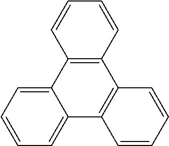 [SMILES: c1ccc2c(c1)c1ccccc1c1ccccc21] | 427 nm | −0.93 eV |
| DIBENZOTHIOPHENE | 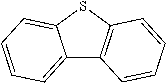 [SMILES: c1ccc2c(c1)sc1ccccc12] | 415 nm | −0.95 eV |
| DIBENZOFURAN | 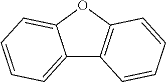 [SMILES: c1ccc2c(c1)oc1ccccc12] | 417 nm | −0.92 eV |
| FLUORENE (REFERENCE EXAMPLE) | 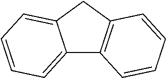 [SMILES: c1ccc2c(c1)Cc1ccccc1-2] | 422 nm | −0.71 eV |
| CHRYSENE (REFERENCE EXAMPLE) | 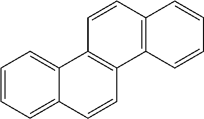 [SMILES: c1ccc2c(c1)ccc1c3ccccc3ccc21] | 500 nm | −1.27 eV |
| ANTHRACENE (REFERENCE EXAMPLE) | 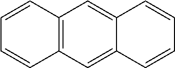 [SMILES: c1ccc2cc3ccccc3cc2c1] | 672 nm | −1.63 eV |

The HOMO and the LUMO of the compound according to the present invention are primarily localized on the central condensed ring. In general, the HOMO and the LUMO of an organic compound strongly relate to excitation or carrier conduction, and a high electrical energy load is applied to a portion of the compound on which the HOMO and the LUMO are localized. Accordingly, since the portion on which the HOMO and the LUMO are localized is required to have chemical stability, a condensed ring including no sp3 carbon atoms which are liable to be oxidized, such as the central condensed ring of the compound according to the present invention, is preferable.

The compound according to the present invention has a high carrier conductivity. Therefore, in particular, when the compound according to the present invention is used as a host material of a light emitting layer, a driving voltage of a device can be decreased. In addition, the central condensed ring of the compound according to the present invention is a condensed ring having a large π conjugated plane. On the other hand, a monocyclic aromatic ring, such as benzene or thiophene, is not preferable.

The properties of the central condensed ring of the compound according to the present invention is reflected in the properties thereof, and in particular, various property values of the T1 energy, the HOMO level, and the LUMO level of the central condensed ring are strongly reflected.

The compound according to the present invention has two m-terphenyl substituents on the central condensed ring thereof. The compound having this structure is preferably used for an organic light emitting device. Hereinafter, the details of this embodiment will be described.

In an organic light emitting device, when the condensed ring as described above is used without being substituted, since the crystallinity of the condensed ring is high, an amorphous film cannot be obtained, and hence, this condensed ring cannot be used for a light emitting device. Accordingly, it has been known that an aryl group or the like is used as a substituent. However, in this case, when π conjugation spreads from the central condensed ring to the aryl group functioning as a substituent, the T1 energy of the whole compound decreases, and a specific T1 energy of the central condensed ring cannot be maintained. The size of the π conjugation between the central condensed ring and the aryl-group substitute is approximately determined by the dihedral angle between these two aromatic rings, that is, between the central condensed ring and the aryl-ring substituent. Therefore, if the dihedral angle can be increased, the π conjugation therebetween can be decreased, and the T1 energy as the molecule can be made close to the T1 energy of the central condensed ring itself. As a result, when a central condensed ring having a high T1 energy is used, this high T1 energy can be maintained.

The present inventors found that it is important that two m-terphenyl groups each functioning as a substituent be bonded at the 2′-position to the central condensed ring. The dihedral angle between the central condensed ring and a benzene-ring substitute (benzene ring located at the center of m-terphenyl) is large. The substitution position number of the m-terphenyl group is shown below.

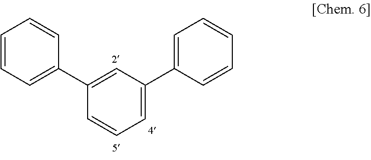 [SMILES: c1ccc(-c2cccc(-c3ccccc3)c2)cc1]

By using a compound having a phenanthrene ring as the central condensed ring by way of example, comparison between substitution positions of the m-terphenyl group was performed using the dihedral angle between the phenanthrene ring and a benzene-ring substitute, which are structure-optimized by molecular orbital calculation, and the results are shown in the following Table 2. In the 2′-position substitution product, two phenyl groups located at the ortho positions with respect to the phenanthrene-phenyl bond each function as a large steric hindrance group, therefore its dihedral angle is remarkably increased. The effect of the remarkable steric hindrance as described above cannot be obtained at the 4′- and the 5′-substitution positions. Therefore, the T1 energy of the 2′-position substitution product is not so much decreased from a high T1 energy of phenanthrene and is maximized. On the other hand, by the 4′-position substituent product, since the π conjugation widely spreads from phenanthrene to a p-biphenyl portion, the T1 energy is seriously decreased.

Besides the phenanthrene ring, the central condensed rings shown in Table 1, that is, a naphthalene ring, a triphenylene ring, a dibenzothiophene ring, and a dibenzofuran ring, also have effects similar to those described above. That is, it can be said that as long as the aromatic rings mentioned above are used, regardless of the types thereof, the T1 energy of the 2′-position substitution product is not so much decreased from a high Ti energy of the central condensed ring and is higher than that of any one of the other substitution products.

TABLE 2

| SUBSTITUTION | | DIHEDRAL |
|---|---|---|
| POSITION | STRUCTURAL FORMULA | ANGLE |
| 2′-POSITION | 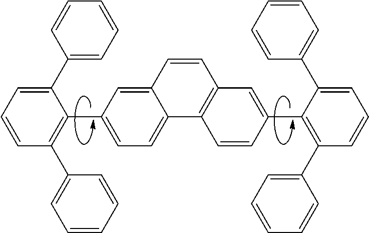 [SMILES: c1ccc(-c2cccc(-c3ccccc3)c2-c2ccc3c(ccc4cc(-c5c(-c6ccccc6)cccc5-c5ccccc5)ccc43)c2)cc1] | 61.6° |
| 4′-POSITION | 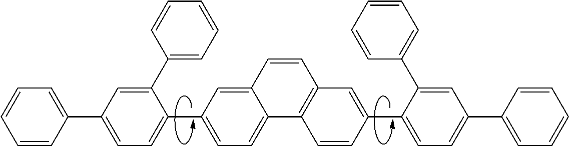 [SMILES: c1ccc(-c2ccc(-c3ccc4c(ccc5cc(-c6ccc(-c7ccccc7)cc6-c6ccccc6)ccc54)c3)c(-c3ccccc3)c2)cc1] | 49.4° |
| 5′-POSITION | 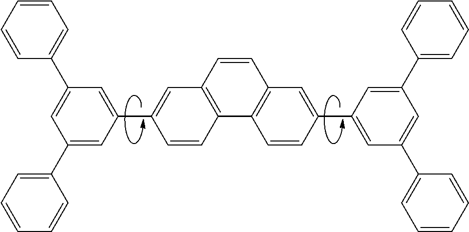 [SMILES: c1ccc(-c2cc(-c3ccccc3)cc(-c3ccc4c(ccc5cc(-c6cc(-c7ccccc7)cc(-c7ccccc7)c6)ccc54)c3)c2)cc1] | 38.9° |

Furthermore, the LUMO level of the compound according to the present invention is deep. The reason for this is that the LUMO level of the central condensed ring is deep and is strongly reflected in the whole compound.

Properties of Organic Light Emitting Device Using m-Terphenyl Compound According to Present Invention

The compound according to the present invention is primarily used for a light emitting layer of an organic light emitting device. Furthermore, besides the light emitting layer, the compound according to the present invention may also be used for any layers, such as a hole injection layer, a hole transport layer, a hole/exciton blocking layer, an electron transport layer, and an electron injection layer.

In this case, the light emitting layer may be formed of a plurality types of components, and the components can be classified into a primary component and at least one accessory component. The primary component is a compound having a highest weight ratio among all compounds forming the light emitting layer and may be called a host material in some cases. The accessory component is a compound other than the primary component and may be called a guest (dopant) material, a light emitting assistant material, and a charge injection material. In this case, the guest material is a compound primarily responsible for light emission in the light emitting layer. On the other hand, the host material is a compound present as a matrix around the guest material in the light emitting layer and is primarily responsible for carrier transportation and supply of excitation energy to the guest material.

The concentration of the guest material to the host material is in a range of 0.01 to 50 percent by weight on the basis of the total weight of constituent materials of the light emitting layer and is preferably in a range of 0.1 to 20 percent by weight. In order to prevent concentration quenching, the concentration of the guest material is more preferably 10 percent by weight or less. In addition, the guest material may be uniformly contained in the whole layer formed of the host material or may be contained so as to have a concentration gradient, and alternatively, the guest material may be partially contained in a specific region so as to form a host material layer region in which no guest material is contained.

The compound according to the present invention is primarily used as a host material of a light emitting layer which uses a phosphorescence material as a guest material. Although the color of the phosphorescence material is not particularly limited in this case, a green light emitting material having a maximum light emission peak wavelength in a range of 500 to 530 nm is preferable.

In a general phosphorescence light emitting device, in order to prevent a decrease in light emitting efficiency caused by non-radiative deactivation from T1 of a host material, the T1 energy thereof must be higher than that of a phosphorescence material functioning as a guest material.

In the compound according to the present invention, since the T1 energy of the central condensed ring is 475 nm or less, the T1 energy of the compound according to the present invention is 490 nm or less and hence is higher than the T1 energy of the green phosphorescence material. Accordingly, when the compound according to the present invention is used as a host material of a green phosphorescence light emitting layer, an organic light emitting device having a high light emitting efficiency can be obtained.

Furthermore, since having a deep LUMO level, when the compound according to the present invention is used as a host material of the light emitting layer, the driving voltage of the device can be decreased. The reason for this is that when the LUMO level is deep, a barrier against electron injection from an electron transport layer or a hole blocking layer adjacent to the light emitting layer at a cathode side can be decreased.

### Examples of m-Terphenyl Compound according to Present Invention

Particular structural formulas of the m-terphenyl compound according to the present invention will be shown below by way of example.

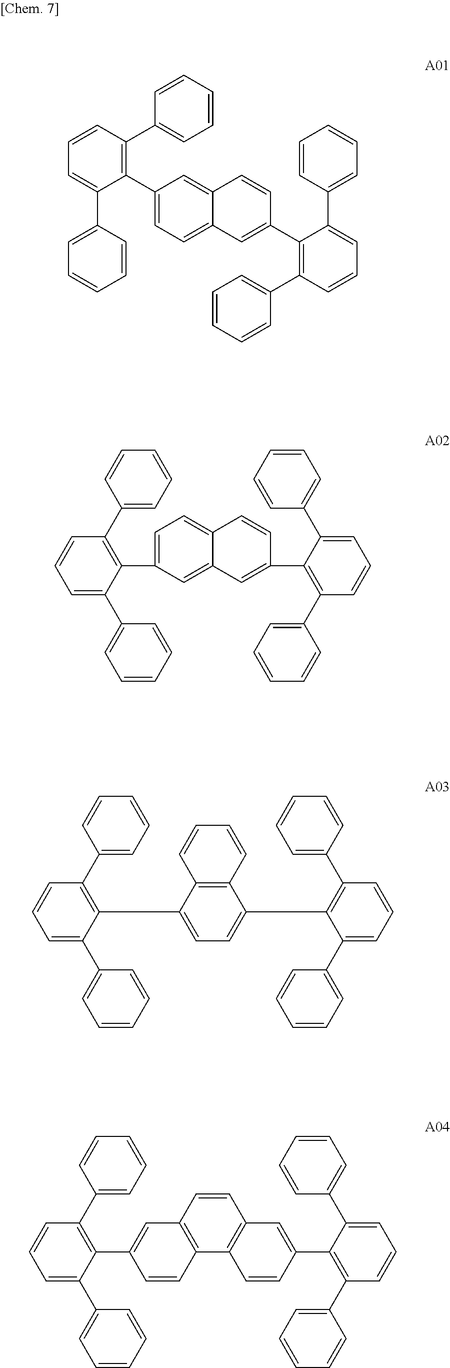

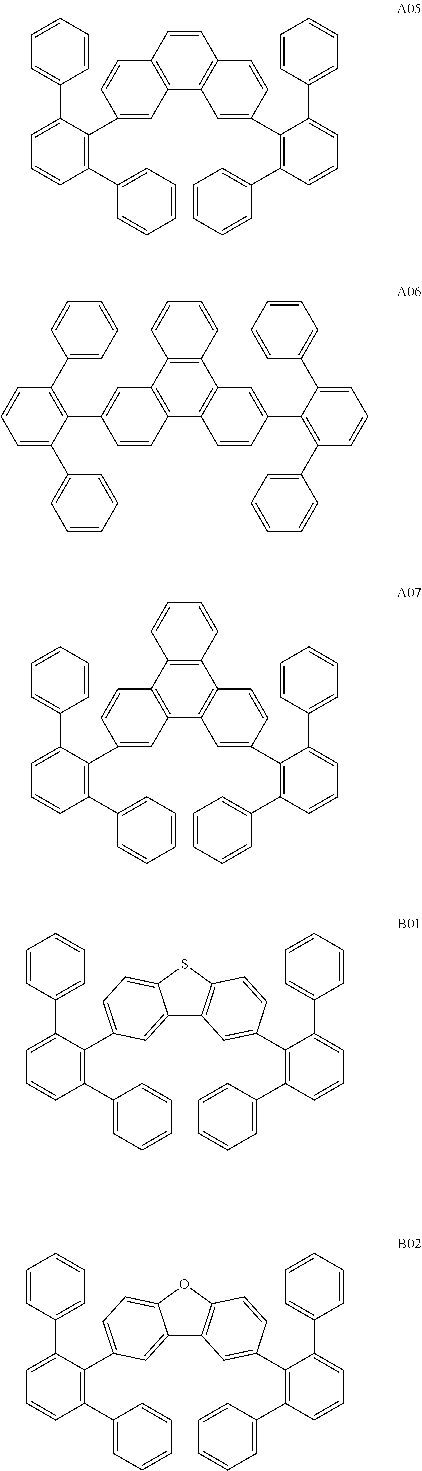

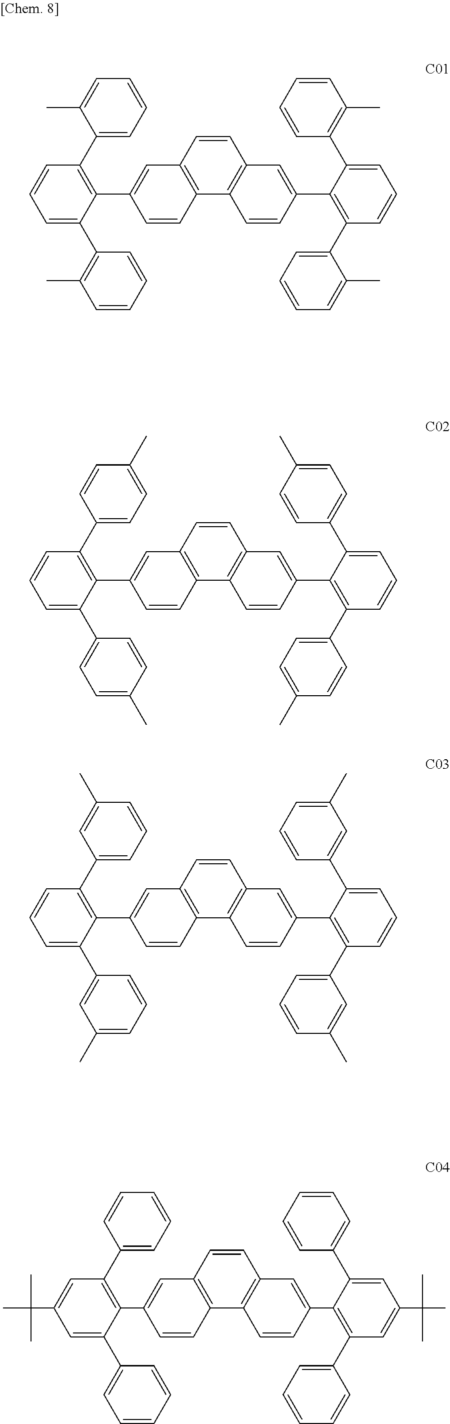

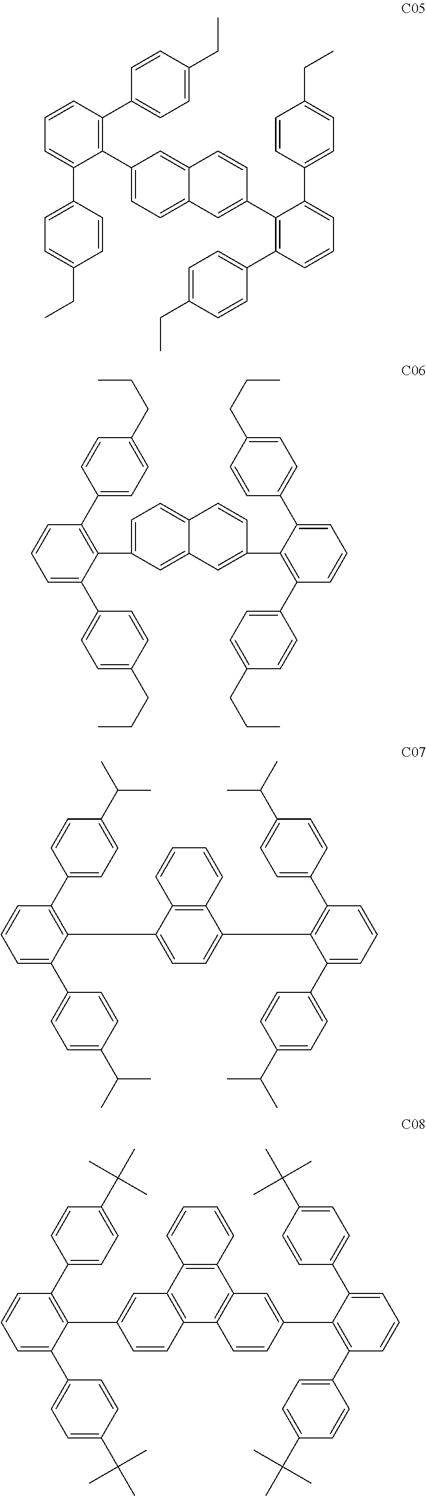

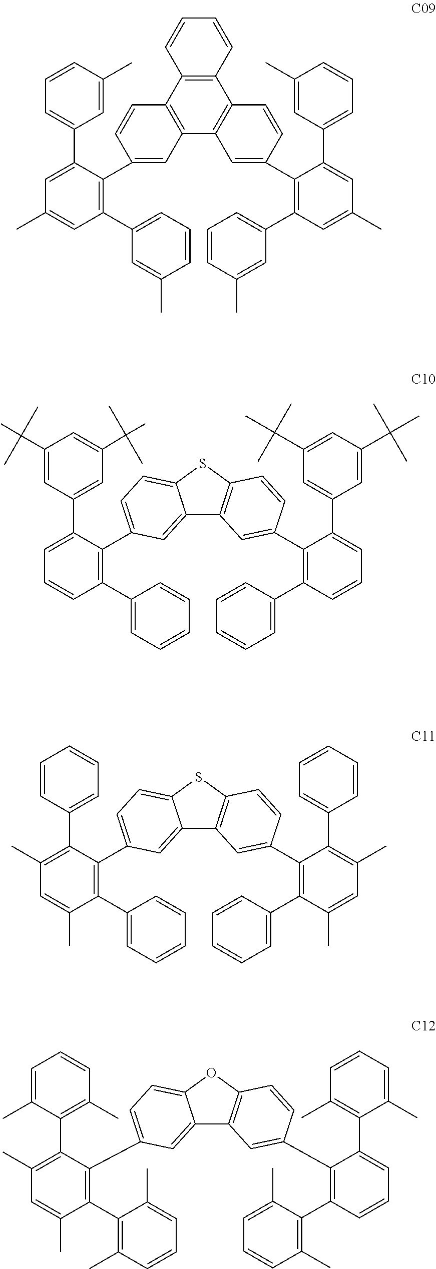

Among the above exemplified compounds, the compounds shown in an A group are each a compound in which the central condensed ring represented by Ar of general formula [1] is a hydrocarbon aromatic ring and in which R₁ to R₂₆ are all hydrogen atoms, and the above compounds are each formed of only hydrogen atoms and sp2 carbon atoms. Therefore, the compounds of the A group each have significantly high chemical stability, and an organic light emitting device which uses one of the above compounds as a host material of a light emitting layer can be expected to have a long life.

Among the above exemplified compounds, the compounds shown in a B group are each a compound in which the central condensed ring represented by Ar of general formula [1] is a dibenzothiophene or dibenzofuran and in which R₁ to R₂₆ are all hydrogen atoms. Since the T1 energy of dibenzothiophene and that of dibenzofuran are particularly high among the central condensed rings represented by Ar, the T1 energy of the compound shown in the B group is very high, such as less than 440 nm. Therefore, the above compound can be satisfactorily used as a host material for a phosphorescence light emitting device in which, besides a green phosphorescence material, a blue phosphorescence material having a maximum light emission wavelength in a range of 440 to 470 nm is used as a guest material.

Among the above exemplified compounds, the compounds shown in a C group are each a compound in which at least one of R₁ to R₂₆ of general formula [1] is an alkyl group having 1 to 4 carbon atoms. Since the solubility of the compound is improved by an alkyl group bonded to the m-terphenyl group by substitution, the compounds shown in the C group are each effectively used when handling properties of a material are improved and/or when an organic light emitting device is formed by a coating process. In addition, since the intermolecular distance of the compound in an amorphous film state is increased by an excluded volume effect of the alkyl group, the compounds shown in the C group are materials each having a lower carrier mobility. Therefore, when it is intended to decrease the carrier mobility in the light emitting layer, the above compounds are each effectively used as a host material thereof.

Synthetic Method of m-Terphenyl Compound According to Present Invention

Next, a synthetic method of the m-terphenyl compound represented by formula [1] according to this embodiment will be described.

The m-terphenyl compound according to the present invention may be synthesized by a coupling reaction between a diboronic acid bis(pinacol) ester compound including the central condensed ring Ar and a 2′-halogenated m-terphenyl compound with a Pd catalyst as shown by the following formula [3].

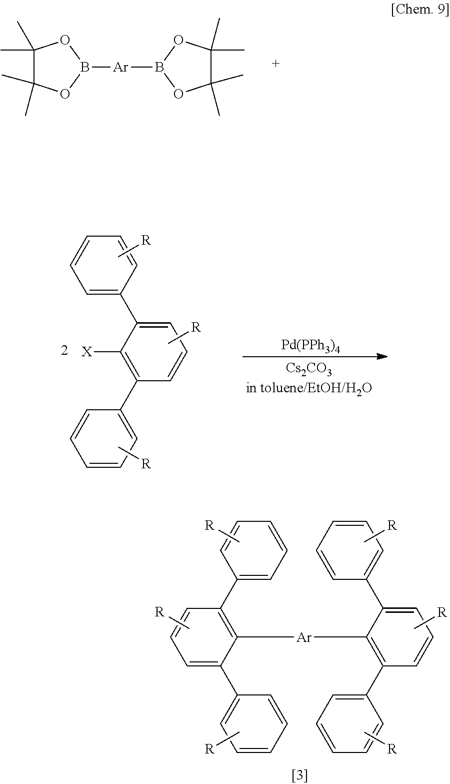

In formula [3], Ar is selected from the arylene groups shown in formula [2]. X indicates bromine or iodine. R indicates a hydrogen atom or an alkyl group having 1 to 4 carbon atoms.

By appropriately selecting Ar and R of the] above reaction, a desired m-terphenyl compound according to the present invention can be synthesized.

In addition, when being used for an organic light emitting device, the compound according to the present invention is preferably processed by sublimation refining as refining performed immediately before the use thereof. The reason for this is that for high purification of an organic compound, sublimation refining has an excellent refining effect. In the sublimation refining described above, in general, a higher temperature is required as the molecular weight of an organic compound is increased, and hence, in this case, thermal decomposition is liable to occur at a high temperature. Therefore, an organic compound used for an organic light emitting device preferably has a molecular weight of 1,000 or less so that sublimation refining can be performed without excessive heating.

### Organic Light Emitting Device According to Present Invention

Next, an organic light emitting device according to the present invention will be described.

The organic light emitting device according to the present invention is a light emitting device which at least includes a pair of electrodes facing each other, that is, an anode and a cathode, and at least one organic compound layer disposed therebetween. Of the at least one organic compound layer, a layer containing a light emitting material is a light emitting layer. In addition, in the organic light emitting device according to the present invention, the organic compound layer contains the m-terphenyl compound represented by general formula [1].

As the organic light emitting device according to the present invention, for example, a device in which an anode, a light emitting layer, and a cathode are provided in this order on a substrate may be mentioned. Besides the above device, for example, a device in which an anode, a hole transport layer, an electron transport layer, and a cathode are provided in this order may also be mentioned. In addition, for example, there may also be mentioned a device in which an anode, a hole transport layer, a light emitting layer, an electron transport layer, and a cathode are provided in this order, and a device in which an anode, a hole injection layer, a hole transport layer, a light emitting layer, an electron transport layer, and a cathode are provided in this order. Furthermore, for example, a device in which an anode, a hole transport layer, a light emitting layer, a hole/exciton blocking layer, an electron transport layer, and a cathode are provided in this order may also be mentioned. However, examples of these five types of multilayer organic light emitting devices simply have very basic device structures, and the structure of an organic light emitting device using the compound according to the present invention is not limited thereto. For example, various lamination structures may be formed in which, for example, an insulating layer is provided at the interface between an electrode and an organic compound layer, an adhesion layer or an interference layer is provided, and an electron transport layer or a hole transport layer is formed of two layers having different ionization potentials.

As the device structure in the above case, a top emission structure in which light is extracted from a substrate-side electrode or a bottom emission structure in which light is extracted from a side opposite to a substrate may be used, and in addition, a dual emission structure may also be used.

Although the m-terphenyl compound according to the present invention may be used in any lamination structures as an organic compound layer of this light emitting device, it is preferably used as the light emitting layer. The m-terphenyl compound according to the present invention is more preferably used as a host material of the light emitting layer and is even more preferably used as a host material of a light emitting layer which contains a phosphorescence material as a guest material.

When the m-terphenyl compound according to the present invention is used as a host material of a phosphorescence light emitting layer, as the phosphorescence material used as a guest material, for example, there may be mentioned metal complexes, such as an iridium complex, a platinum complex, a rhenium complex, a copper complex, a europium complex, and a ruthenium complex. Among these mentioned above, an iridium complex which exhibits strong phosphorescence properties is preferably used. In addition, in order to assist transfer of excitons or carriers, the light emitting layer may contain a plurality of phosphorescence materials.

Although particular examples of an iridium complex used as the phosphorescence material of the present invention will be shown below, the present invention is not limited thereto.

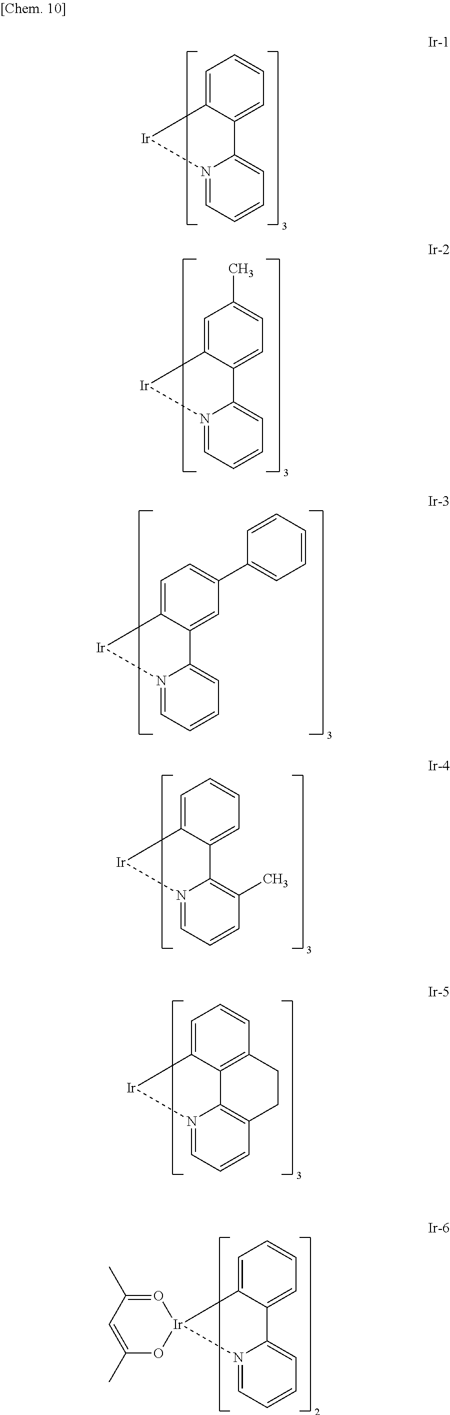

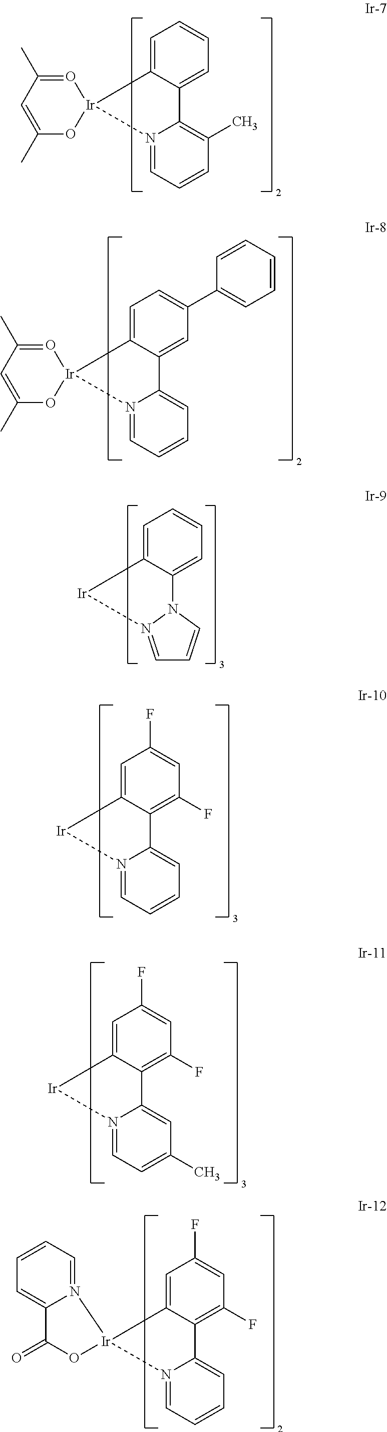

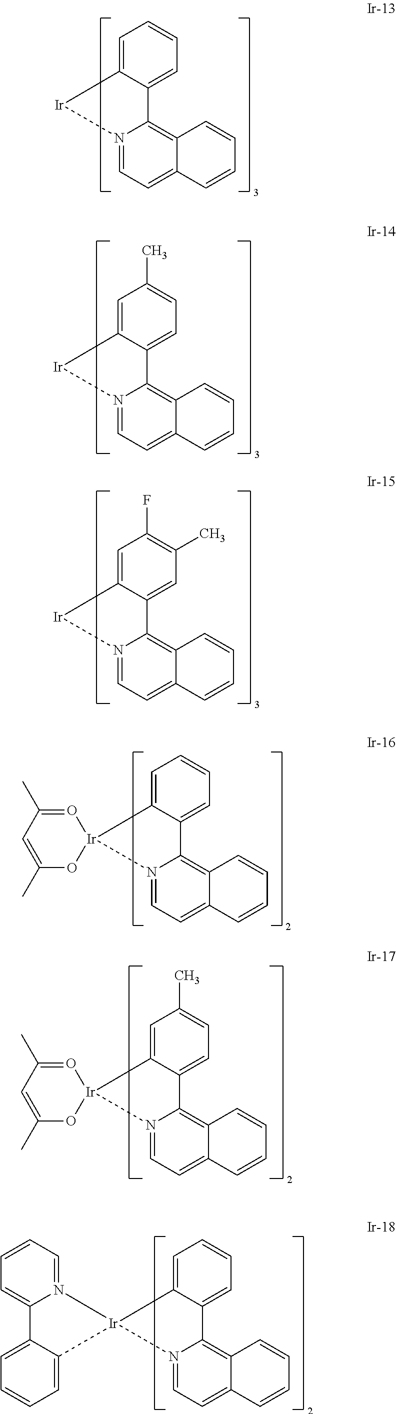

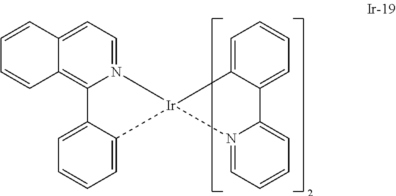

In this embodiment, besides the compounds according to the present invention, if needed, known low molecular weight and high molecular weight compounds may also used. In more particular, for example, at least one of a hole injection material, a hole transport material, a host material, a light emitting material, an electron injection material, and an electron transport material may also be used together with the compound according to the present invention.

Hereinafter, examples of these compounds will be described.

As the hole injection transport material, a material into which holes from an anode is easily injected and which has a high hole mobility so as to be able to transport injected holes to a light emitting layer is preferable. As a low molecular weight and a high molecular weight material which have hole injection/transport properties, for example, a triarylamine derivative, a phenylenediamine derivative, a stilbene derivative, a phthalocyanine derivative, a porphyrin derivative, a polyvinylcarbazole, a polythiophene, and other conductive polymers may be mentioned.

As the light emitting material primarily responsible for a light emitting function, besides the phosphorescence guest materials mentioned above or derivatives thereof, for example, there may be mentioned condensed ring compounds (such as a fluorene derivative, a naphthalene derivative, a pyrene derivative, a perylene derivative, a tetracene derivative, an anthracene derivative, and rubrene), a quinacridone derivative, a coumarin derivative, a stilbene derivative, an organic aluminum complex, such as tris(8-quinolinolate)aluminum, an organic beryllium complex, and polymer derivatives, such as a polyphenylenevinylene derivative, a polyfluorene derivative, and a polyphenylene derivative.

As the electron injection transport material, a material into which electrons from a cathode are easily injected and which is able to transport injected electrons to a light emitting layer may be arbitrarily selected in consideration, for example, of the balance with the hole mobility of the hole injection transport material. As a material having electron injection transport properties, for example, there may be mentioned an oxadiazole derivative, an oxazole derivative, a pyrazine derivative, a triazole derivative, a triazine derivative, a quinoline derivative, a quinoxaline derivative, a phenanthroline derivative, and an organic aluminum complex.

As an anode material, a material having a work function as high as possible is preferably used. For example, there may be mentioned a metal element, such as gold, platinum, silver, copper, nickel, palladium, cobalt, selenium, vanadium, or tungsten; an alloy containing at least two of the metals mentioned above; or a metal oxide, such as tin oxide, zinc oxide, indium oxide, indium tin oxide (ITO), or indium zinc oxide. In addition, a conductive polymer, such as a polyaniline, a polypyrrole, or a polythiophene, may also be used. These electrode materials may be used alone or in combination. In addition, the anode may have either a monolayer structure or a multilayer structure.

On the other hand, as a cathode material, a material having a low work function is preferably used. For example, there may be mentioned an alkali metal, such as lithium, an alkaline earth metal, such as calcium, or a metal element, such as aluminum, titanium, manganese, silver, lead, or chromium. In addition, an alloy containing at least two of the metal elements mentioned above may also be used. For example, magnesium-silver, aluminum-lithium, or aluminum-magnesium may be used. In addition, a metal oxide, such as indium tin oxide (ITO), may also be used. These electrode materials may be used alone or in combination. In addition, the cathode may have either a monolayer structure or a multilayer structure.

In the organic light emitting device according to this embodiment, layers each containing the organic compound according to this embodiment and layers each containing another organic material are formed by the following method. In general, for thin film formation, for example, there may be used a vacuum deposition method, an ionization deposition method, a sputtering method, a plasma deposition method, or a known coating method, such as a spin coating method, a dipping method, a casting method, a Langmuir-Blodgett (LB) method, or an ink jet method, which uses a suitable solvent for forming a solution. When the layer is formed by a vacuum deposition method, a solution coating method, or the like, for example, crystallization is not likely to occur, and excellent stability with time can be obtained. When the layer is formed by a coating method, a film may also be formed in combination with a suitable binder resin.

As the binder resin mentioned above, for example, a polyvinylcarbazole resin, a polycarbonate resin, a polyester resin, an ABS resin, an acrylic resin, a polyimide resin, a phenol resin, an epoxy resin, a silicone resin, or a urea resin may be mentioned; however, the binder resin is not limited thereto. In addition, as the binder resins mentioned above, a homopolymer or a copolymer may be used alone or in combination. Furthermore, if necessary, known additives, such as a plasticizer, an antioxidant, and an ultraviolet absorber, may also be used together with the binder resin.

### Application of Organic Light Emitting Device

The organic light emitting device according to the present invention may be used for a display apparatus or a lighting apparatus. Besides the above application, the organic light emitting device according to the present invention may also be used for an exposure light source of an electrophotographic image forming apparatus, a backlight of a liquid crystal display apparatus, and the like.

A display apparatus has the organic light emitting device according to the present invention at a display portion. This display portion has a plurality of pixels, and each pixel has the organic light emitting device according to the present invention. The display apparatus may be used as an image display apparatus of a personal computer (PC) or the like.

The display apparatus may be used for a display portion of an imaging apparatus, such as a digital camera or a digital video camera. The imaging apparatus has the display portion and an imaging portion having an imaging optical system for image pickup.

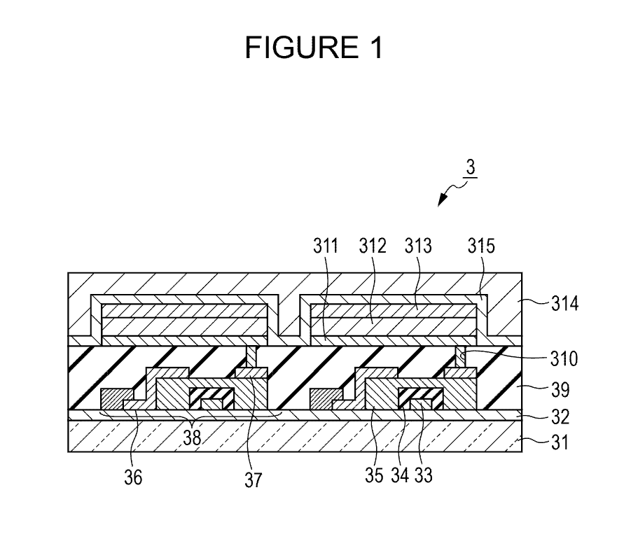

FIG. 1 is a schematic cross-sectional view of an image display apparatus having an organic light emitting device in a pixel portion. In this FIGURE, two organic light emitting devices and two thin film transistors (TFTs) are shown. One organic light emitting device is connected to one TFT.

In the FIGURE, reference numeral 3 indicates an image display apparatus, reference numeral 38 indicates a TFT element which is a switching element, reference numeral 31 indicates a substrate, reference numeral 32 indicates a moisture preventing film, reference numeral 33 indicates a gate electrode, reference numeral 34 indicates a gate insulating film, reference numeral 35 indicates a semiconductor layer, reference numeral 36 indicates a drain electrode, reference numeral 37 indicates a source electrode, and reference numeral 39 indicates an insulating film. In addition, reference numeral 310 indicates a contact hole, reference numeral 311 indicates an anode, reference numeral 312 indicates an organic layer, reference numeral 313 indicates a cathode, reference numeral 314 indicates a first protective layer, and reference numeral 315 indicates a second protective layer.

In the image display apparatus 3, the moisture preventing film 32 is provided on the substrate 31 of a glass or the like in order to protect members (the TFT or the organic layer) formed thereon. As a material forming the moisture preventing film 32, for example, silicon oxide or a compound containing silicon oxide and silicon nitride may be used. The gate electrode 33 is provided on the moisture preventing film 32. The gate electrode 33 is obtained by forming a film of a metal, such as Cr, by sputtering.

The gate insulating film 34 is disposed so as to cover the gate electrode 33. The gate insulating film 34 is formed by the steps of depositing a film of silicon oxide or the like by a plasma CVD method or a catalytic chemical vapor deposition method (cat-CVD method) and patterning the film thus deposited. The semiconductor layer 35 is provided so as to cover the gate insulating film 34 which is patterned and provided on each region in which a TFT is to be formed. This semiconductor layer 35 is formed by the steps of forming a silicon film by a plasma CVD method or the like (depending on the case, followed by performing annealing at a temperature, for example, of 290° C. or more) and patterning this silicon film to form a circuit.

Furthermore, the drain electrode 36 and the source electrode 37 are provided on each semiconductor layer 35. As described above, the TFT element 38 has the gate electrode 33, the gate insulating layer 34, the semiconductor layer 35, the drain electrode 36, and the source electrode 37. The insulating film 39 is provided over the TFT element 38. Next, the contact hole (through hole) 310 is provided in the insulating film 39, and the anode 311 of a metal for an organic light emitting device and the source electrode 37 are connected to each other.

On this anode 311, one or more organic layers 312 containing at least one light emitting layer and the cathode 313 are sequentially laminated so as to form the organic light emitting device functioning as a pixel.

In order to prevent degradation of the organic light emitting device, the first protective layer 314 and/or the second protective layer 315 may be provided.

In addition, the switching element is not particularly limited, and besides the TFT element described above, a metal-insulator-metal (MIM) element may also be used.

### EXAMPLES

### Example 1

### Synthesis of Exemplified Compound A04

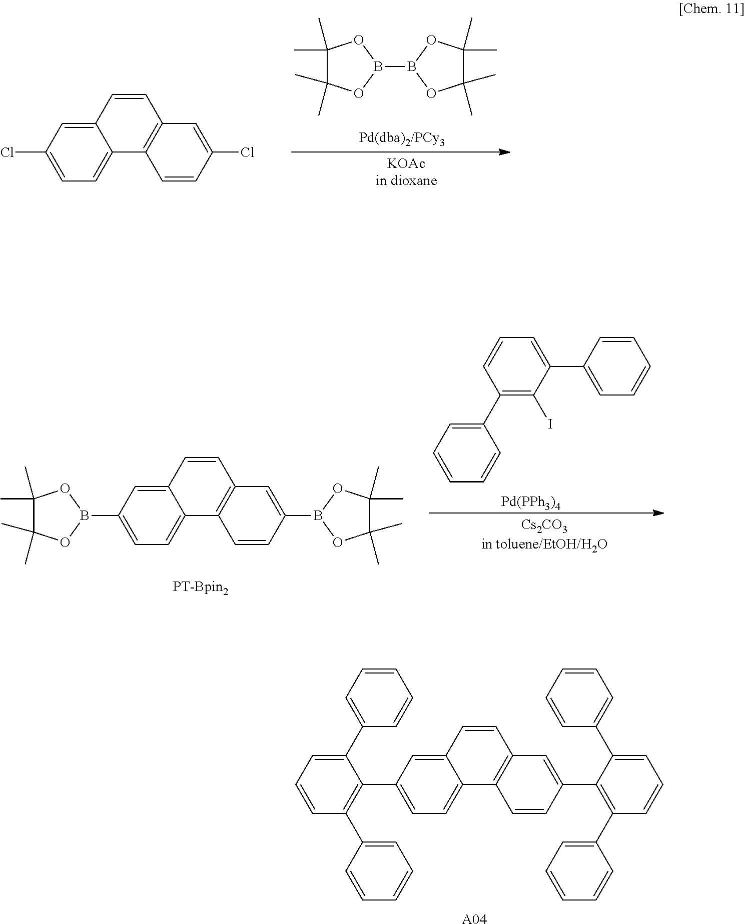

The following reagents and solvent were charged into a 100-ml recovery flask.
2,7-Dichlorophenanthrene: 760 mg (3.08 mmol)
Bis(pinacolato)diboron: 1.95 g (7.69 mmol)
Bis(dibenzylideneacetone)palladium(0):177 mg (0.31 mmol)
Tricyclohexylphosphine: 259 mg (0.92 mmol)
Potassium acetate: 0.60 g (6.11 mmol)
1,4-Dioxane: 32 mL

This reaction solution was stirred at 90° C. for 7 hours in a nitrogen atmosphere. After the reaction was completed, the reaction solution was washed with water and was then dried over sodium sulfate, followed by concentration, so that a crude product was obtained. Next, this crude product was refined using a silica gel column chromatography (eluent: toluene), and 1.02 g of intermediate PT-Bpin₂ was obtained (yield: 77%).

Subsequently, the following reagents and solvents were charged into a 200-mL recovery flask.
2′-Iodo-m-terphenyl: 2.11 g (5.92 mmol)
PT-Bpin₂: 1.02 g (2.37 mmol)
Tetrakis(triphenylphosphine)palladium(0): 137 mg (0.12 mmol)
Toluene: 40 mL
Ethanol: 20 mL
30-wt % cesium carbonate aqueous solution: 20 mL

This reaction solution was heat-refluxed for 3 hours while being stirred in a nitrogen atmosphere. After the reaction was completed, the reaction solution was added with water and was then stirred, and a precipitated crystal was filtrated, followed by washing with water, ethanol, and acetone, so that a crude product was obtained. Next, after this crude product was heated and dissolved in chlorobenzene, this solution thus obtained was filtrated while being hot, and recrystallization was performed twice using chlorobenzene as a solvent. After the crystal thus obtained was vacuum dried at 150° C., sublimation refining was performed at a pressure of 10⁻⁴ Pa and a temperature of 340° C., so that 834 mg of a high-purity exemplified compound A04 was obtained (yield: 55%).

Identification of the obtained compound was performed by a mass analysis.

### [MALDI-TOF-MS (Matrix-Assisted Laser Desorption Ionization Time-of-Flight Mass Spectrometry)]

Observed value: m/z=634.31, Calculated value: C₅₀H₃₄=634.27

In addition, the T1 energy of the exemplified compound A04 was measured by the following method.

A phosphorescence spectrum of a dilute toluene solution of the exemplified compound A04 was measured at 77K and at an excitation wavelength of 350 nm in an Ar atmosphere. From the peak wavelength of the first light emission peak of the obtained phosphorescence spectrum, the T1 energy (equivalent wavelength) was 468 nm.

Next, the energy gap of the exemplified compound A04 was measured by the following method.

The exemplified compound A04 was deposited on a glass substrate by heating, so that a deposition thin film having a thickness of 20 nm was obtained. An absorption spectrum of this deposition thin film was measured using an ultraviolet and visible spectrophotometer (V-560 manufactured by JASCO Corp.). The absorption edge of the obtained absorption spectrum was 347 nm, and the energy gap of the exemplified compound A04 was 3.57 eV.

Furthermore, the ionization potential of the exemplified compound A04 was measured by the following method.

The ionization potential was measured using the deposition thin film used for the above energy gap measurement by a photoelectron spectrometer AC-3 (manufactured by Riken Keiki Co., Ltd.). According to the measurement result, the ionization potential of the exemplified compound A04 was 6.43 eV.

Furthermore, the LUMO level of a compound can be estimated from the difference between an ionization potential value and an energy gap value, and when the LUMO level of the exemplified compound A04 was estimated from the above ionization potential value and energy gap value, −2.86 eV was obtained.

### Example 2

### Synthesis of Exemplified Compound A06

### (1) Synthesis of Intermediate TRP-Bpin₂

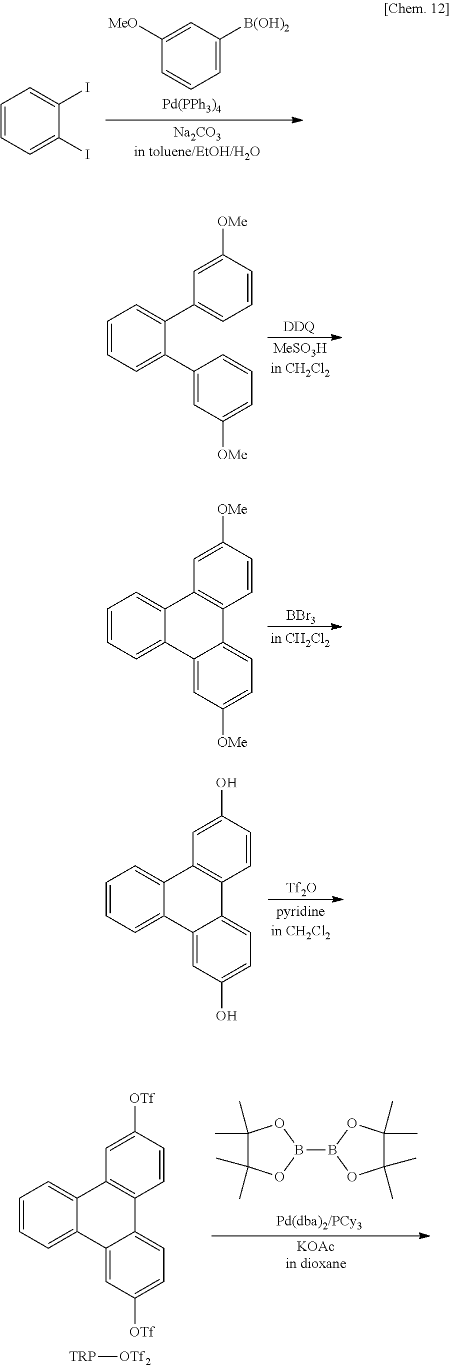

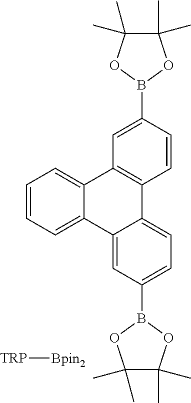

The following reagents and solvents were charged into a 300-mL recovery flask.
1,2-Diiodobenzene: 5.40 g (16.4 mmol)
3-Methoxy-phenylboronic acid: 5.22 g (34.3 mmol)
Tetrakis(triphenylphosphine)palladium(0): 0.70 g (0.61 mmol)
Toluene: 100 mL
Ethanol: 50 mL
10-wt % sodium carbonate aqueous solution: 50 mL

This reaction solution was heat-refluxed for 6 hours while being stirred in a nitrogen atmosphere. After the reaction was completed, the reaction solution was washed with water and was then dried over sodium sulfate, followed by concentration, so that a crude product was obtained. Next, this crude product was refined by a silica gel column chromatography (eluent: heptane/chloroform=1/1), so that 2.29 g of 1,2-bis(3-methoxyphenyl)benzene was obtained (yield: 48%).

Subsequently, the following reagents and solvent were charged into a 300-mL recovery flask.
1,2-Bis(3-methoxyphenyl)benzene: 1.73 g (5.98 mmol)
Dichloromethane: 108 mL,
Methanesulfonic acid: 12 mL

After this mixed solution was added with 2.03 g (8.96 mmol) of 2,3-dichloro-5,6-cyano-p-benzoquinone (DDQ) at 0° C. and was then stirred for 30 minutes at 0° C., the reaction was stopped by addition of a sodium carbonate aqueous solution. Next, after being washed with water and dried over magnesium sulfate, this reaction solution was concentrated, so that a crude product was obtained. Next, this crude product was refined by a silica gel column chromatography (eluent: heptane/toluene=1/1), so that 1.00 g of 2,7-dimethoxytriphenylene was obtained (yield: 58%).

In addition, the compound thus obtained was identified by a ¹H-NMR analysis.

[¹H-NMR (400 MHz, CDCl₃)]

δ 8.58 (dd, 2H), 8.47 (d, 2H), 8.03 (d, 2H), 7.66 (dd, 2H), 7.26 (t, 2H), 4.03 (s, 6H).

Subsequently, the following reagent and solvent were charged into a 100-mL recovery flask.
2,7-Dimethoxytriphenylene: 800 mg (2.77 mmol)
Dichloromethane: 40 mL

This solution was added with 5.83 mL (5.83 mmol) of a 1M dichloromethane solution of boron tribromide at 0° C. and was then stirred for 30 minutes at 0° C. Furthermore, after stirring was performed at room temperature for 6 hours, the reaction was stopped by addition of methanol. A white precipitate thus obtained was filtrated and refined by heating dispersion washing using a mixed solvent containing ethanol and heptane, so that 687 mg of 2,7-dihydroxytriphenylene was obtained (yield: 95%).

Next, the following reagent and solvents were charged into a 100-mL recovery flask equipped with a dropping funnel.
2,7-Dihydroxytriphenylene: 700 mg (2.69 mmol)
Dichloromethane: 38 mL
Pyridine: 1.7 mL

To this solution was added dropwise a mixed solution containing 1.20 mL (7.14 mmol) of trifluoromethanesulfonic anhydride and 3 mL of dichloromethane at 0° C. for 5 minutes. After stirring was further performed for 1 hour at 0° C., the reaction was stopped by addition of water. Next, after being washed with water and dried over sodium sulfate, this reaction solution was concentrated, so that a crude product was obtained. In addition, refining was further performed by heating dispersion washing using ethanol as a solvent, and 1.32 g of intermediate TRP-OTf₂ was obtained (yield: 94%).

Subsequently, the following reagents and solvent were charged into a 100-mL recovery flask.
Intermediate TRP-OTf₂: 833 mg (1.49 mmol)
Bis(pinacolato)diboron: 713 mg (3.28 mmol)
Bis(dibenzylideneacetone)palladium(0): 86 mg (0.15 mmol)
Tricyclohexylphosphine: 126 mg (0.45 mmol)
Potassium acetate: 439 mg (4.47 mmol)
1,4-Dioxane: 30 mL

This reaction solution was stirred at 90° C. for 4 hours in a nitrogen atmosphere. After the reaction was completed, the reaction solution was washed with water and was then dried over sodium sulfate, followed by concentration, so that a crude product was obtained. Next, this crude product was refined by a silica gel column chromatography (eluent: toluene/ethyl acetate), and 453 mg of intermediate TRP-Bpin₂ was obtained (yield: 63%).

### (2) Synthesis of Exemplified Compound A06

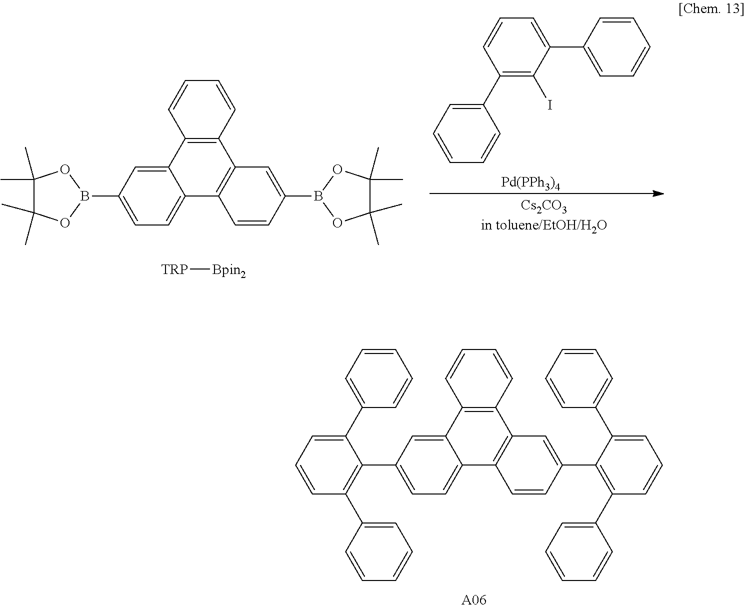

The following reagents and solvents were charged into a 100-mL recovery flask.
2′-Iodo-m-terphenyl: 925 mg (2.60 mmol)
TRP-Bpin₂: 453 mg (0.943 mmol)
Tetrakis(triphenylphosphine)palladium(0): 80 mg (69 μmol)
Toluene: 20 mL
Ethanol: 10 mL
30-wt % cesium carbonate aqueous solution: 10 mL

This reaction solution was heat-refluxed for 3.5 hours while being stirred in a nitrogen atmosphere. After the reaction was completed, the reaction solution was added with water and was stirred, and a precipitated crystal was then filtrated, followed by performing washing with water, ethanol, and acetone, so that a crude product was obtained. Next, after this crude product was heated and dissolved in toluene, this solution thus obtained was filtrated while being hot, and recrystallization was then performed using toluene as a solvent. After the crystal thus obtained was vacuum dried at 150° C., sublimation refining was performed at a pressure of 10⁻⁴ Pa and a temperature of 360° C., so that 251 mg of a high-purity exemplified compound A06 was obtained (yield: 39%).

The result of identification of the obtained compound is shown below.

### [MALDI-TOF-MS]

Observed value: m/z=684.35, Calculated value: C₅₄H₃₆=684.28

[¹H-NMR (400 MHz, CDCl₃)]

δ 8.07 (d, 2H), 8.04 (d, 2H), 7.99 (dd, 2H), 7.57-7.45 (m, 6H), 7.41 (dd, 2H), 7.18-7.00 (m, 22H).

In addition, the T1 energy (equivalent wavelength) of the exemplified compound A06 was 469 nm measured by a method similar to that of Example 1.

When the energy gap of the exemplified compound A06 was further measured by a method similar to that of Example 1, the absorption edge of the absorption spectrum was 363 nm, and the energy gap of the exemplified compound A06 was 3.42 eV.

When the ionization potential of the exemplified compound A06 was further measured by a method similar to that of Example 1, the ionization potential of the exemplified compound A06 was 6.34 eV.

Furthermore, when the LUMO level of the exemplified compound A06 was estimated by a method similar to that of Example 1, it was estimated to be −2.92 eV.

### Example 3

### Synthesis of Exemplified Compound B01

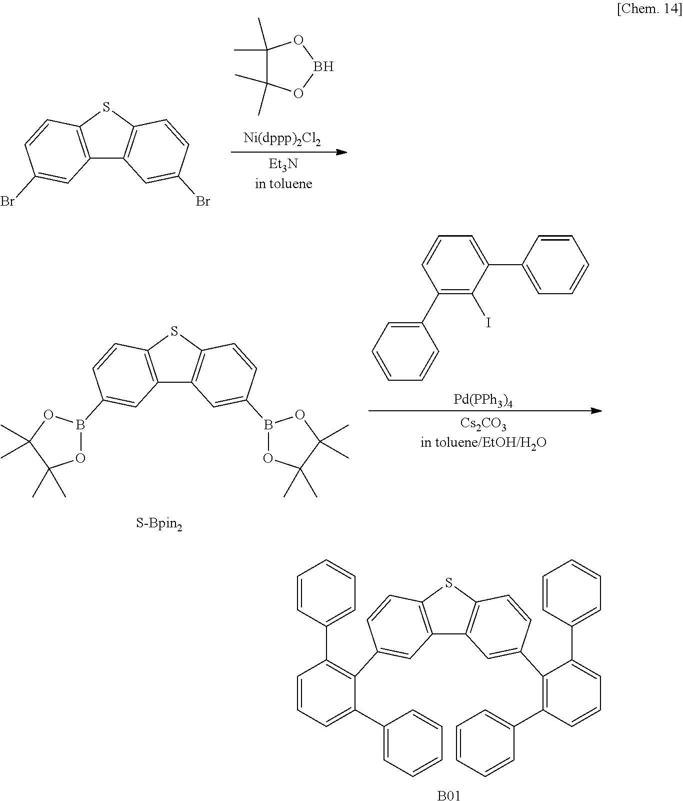

The following reagents and solvents were charged into a 100-mL recovery flask.
2,8-Dibromodibenzothiophene: 600 mg (1.75 mmol)
(1,3-Bis(diphenylphosphino)propane)dichloronickel(II): 380 mg (0.70 mmol)
Toluene: 24 mL
Triethylamine: 1.46 mL

After this reaction solution was heated to 90° C. and was then added with 1.53 mL (10.5 mmol) of 4,4,5,5-tetramethyl-1,3,2-dioxaborolane, heating was performed for 6.5 hours. After the reaction solution was cooled to room temperature, the reaction was quenched by addition of water, and an insoluble matter was removed by filtration of the reaction solution. After a reaction product in the filtrate was extracted using toluene, this extracted solution was washed with water and was then dried over sodium sulfate, followed by concentration, so that a crude product was obtained. The obtained crude product was refined by a silica gel column chromatography (eluent: toluene), and 124 mg of intermediate S-Bpin₂ was obtained (yield: 16%).

Then, the following reagents and solvents were charged into a 50-mL recovery flask.
2′-Iodo-m-terphenyl: 245 mg (0.688 mmol)
S-Bpin₂:120 mg (0.275 mmol)
Tetrakis(triphenylphosphine)palladium(0): 25 mg (22 μmol)
Toluene: 6 mL
Ethanol: 3 mL
30-wt % cesium carbonate aqueous solution: 3 mL

This reaction solution was heat-refluxed for 4.5 hours while being stirred in a nitrogen atmosphere. After the reaction was completed, the reaction solution was washed with water and was then dried over sodium sulfate, followed by concentration, so that a crude product was obtained. Next, this crude product was refined by a silica gel column chromatography (eluent: heptane/chloroform=3/1), and heating dispersion washing was further performed using a mixed solvent containing hexane and ethanol. After a crystal thus obtained was vacuum dried at 150° C., sublimation refining was performed at a pressure of 10⁻⁴ Pa and a temperature of 340° C., so that 120 mg of a high-purity exemplified compound B01 was obtained (yield: 68%).

The result of identification of the obtained compound is shown below.

### [MALDI-TOF-MS]

Observed value: m/z=640.23, Calculated value: C₄₈H₃₂=640.22

[¹H-NMR (400 MHz, CDCl₃)]

δ 7.75-7.42 (m, 6H), 7.35 (d, 2H), 7.16-7.01 (m, 22H), 6.85 (dd, 2H).

In addition, the T1 energy (equivalent wavelength) of the exemplified compound B01 was 423 nm measured by a method similar to that of Example 1.

When the energy gap of the exemplified compound B01 was further measured by a method similar to that of Example 1, the absorption edge of the absorption spectrum was 322 nm, and the energy gap of the exemplified compound B01 was 3.85 eV.

In addition, when the ionization potential of the exemplified compound B01 was measured by a method similar to that of Example 1, 6.49 eV was obtained.

Furthermore, when the LUMO level of the exemplified compound B01 was estimated by a method similar to that of Example 1, it was estimated to be −2.64 eV.

### Example 4

### Synthesis of Exemplified Compound C02

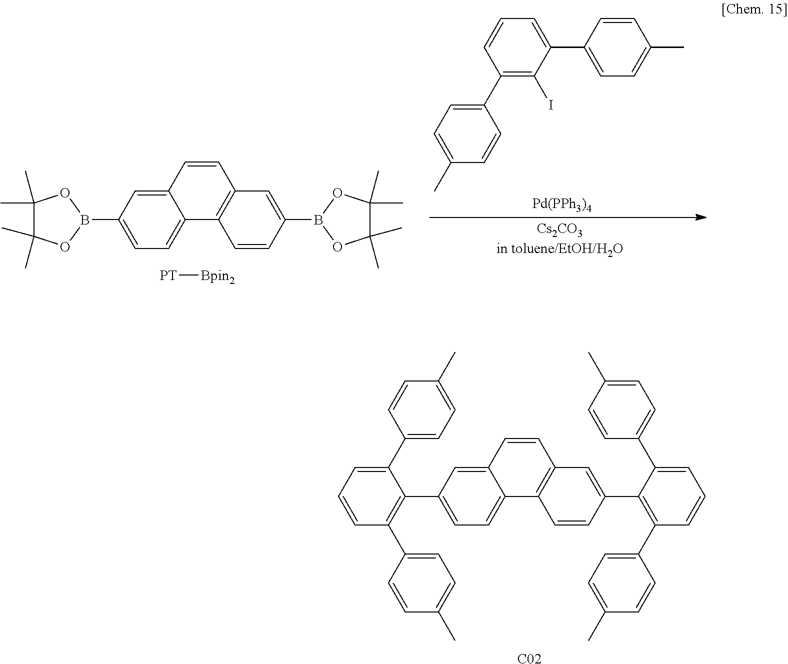

The following reagents and solvents were charged into a 50-mL recovery flask.
4,4″-Dimethyl-2′-iodo-m-terphenyl: 1.12 g (2.91 mmol)
PT-Bpin₂: 500 mg (1.16 mmol)
Tetrakis(triphenylphosphine)palladium(0): 67 mg (58 μmol)
Toluene: 20 mL
Ethanol: 10 mL
30-wt % cesium carbonate aqueous solution: 10 mL

This reaction solution was heat-refluxed for 5 hours while being stirred in a nitrogen atmosphere. After the reaction was completed, the reaction solution was added with water and was stirred, and a precipitated crystal was filtrated and was then washed with water, ethanol, and acetone, so that a crude product was obtained. Next, after this crude product was heated and dissolved in toluene, this solution thus obtained was filtrated while being hot, and recrystallization was performed twice using toluene as a solvent. After the obtained crystal was vacuum dried at 150° C., sublimation refining was performed at a pressure of 10⁻⁴ Pa and a temperature of 350° C., so that 418 mg of a high-purity exemplified compounds C02 was obtained (yield: 52%).

The obtained compound was identified by a mass analysis.

### [MALDI-TOF-MS (Matrix-Assisted Laser Desorption Ionization Time-of-Flight Mass Spectrometry)]

Observed value: m/z=690.45, Calculated value: C₅₄H₄₂=690.33

In addition, the T1 energy (equivalent wavelength) of the exemplified compound C02 was 469 nm measured by a method similar to that of Example 1.

When the energy gap of the exemplified compound C02 was further measured by a method similar to that of Example 1, the absorption edge of the absorption spectrum was 349 nm, and the energy gap of the exemplified compound C02 was 3.55 eV.

In addition, when the ionization potential of the exemplified compound C02 was further measured by a method similar to that of Example 1, it was 6.40 eV.

Furthermore, when the LUMO level of the exemplified compound C02 was estimated by a method similar to that of Example 1, it was estimated to be −2.85 eV.

### Comparative Example 1

### Comparison of T1 Energy, Ionization Potential, and Estimated Value of LUMO Level

The T1 energy and the ionization potential of each of comparative compounds H01 to H04 shown below were measured by a method similar to that of Example 1, and furthermore, the LUMO level thereof was estimated from the values obtained by the above measurement. The results are shown in Table 3 together with the results of Examples 1 to 4.

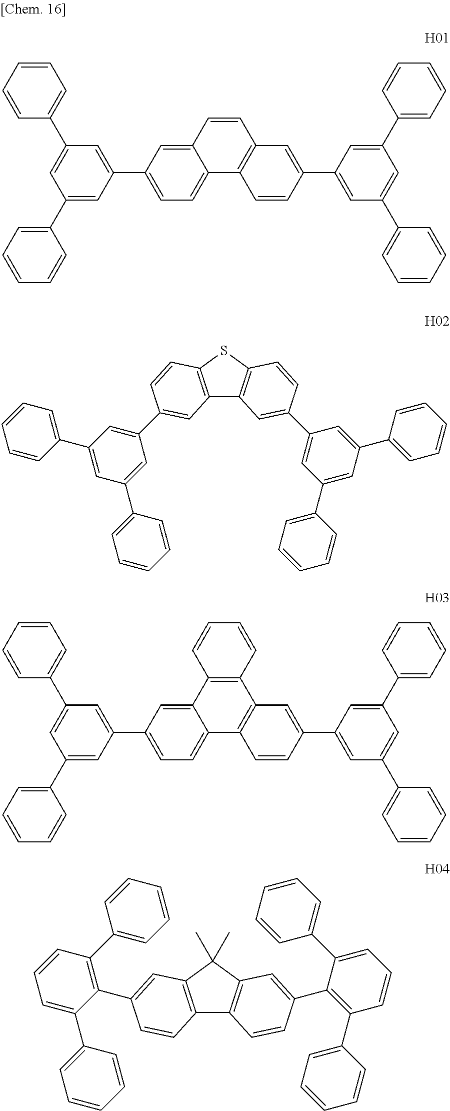

TABLE 3

| | | T1 ENERGY | | | LUMO LEVEL |
|---|---|---|---|---|---|
| | CENTRAL | (EQUIVALENT | ENERGY | IONIZATION | (ESTIMATED |
| | CONDENSED RING | WAVELENGTH) | GAP | POTENTIAL | VALUE) |
| EXEMPLIFIED | PHENANTHRENE | 468 nm | 3.57 eV | 6.43 eV | −2.86 eV |
| COMPOUND A04 | | | | | |
| EXEMPLIFIED | PHENANTHRENE | 469 nm | 3.55 eV | 6.40 eV | −2.85 eV |
| COMPOUND C02 | | | | | |
| COMPARATIVE | PHENANTHRENE | 480 nm | 3.38 eV | 6.20 eV | −2.82 eV |
| COMPOUND H01 | | | | | |
| EXEMPLIFIED | TRIPHENYLENE | 469 nm | 3.42 eV | 6.34 eV | −2.92 eV |
| COMPOUND A06 | | | | | |
| COMPARATIVE | TRIPHENYLENE | 487 nm | 3.25 eV | 6.06 eV | −2.81 eV |
| COMPOUND H03 | | | | | |
| EXEMPLIFIED | DIBENZOTHIOPHENE | 423 nm | 3.85 eV | 6.49 eV | −2.64 eV |
| COMPOUND B01 | | | | | |
| COMPARATIVE | DIBENZOTHIOPHENE | 449 nm | 3.62 eV | 6.16 eV | −2.54 eV |
| COMPOUND H02 | | | | | |
| COMPARATIVE | FLUORENE | 483 nm | 3.50 eV | 6.27 eV | −2.77 eV |
| COMPOUND H04 | | | | | |

When the comparative compound H01 was compared to the exemplified compounds A04 and C02, the compound H01 being a structural isomer of the compound A04 having a phenanthrene ring as the central condensed ring, and the compound C02 being a derivative of the compound A04, the exemplified compounds A04 and C02 according to the present invention had a higher T1 energy and a deeper estimated value of the LUMO level (the absolute value is high). As in the case described above, between the exemplified compound A06 and the comparative compound H03, each having a triphenylene ring as the central condensed ring, and between the exemplified compound B01 and the comparative compound H02, each having a dibenzothiophene ring as the central condensed ring, the compounds according to the present invention had a higher T1 energy and a deeper estimated value of the LUMO level. In addition, when the comparative compound H04 having a fluorene ring as the central condensed ring is compared to the exemplified compound A04 and A06 of the present invention, each having the same 2′-m-terphenyl substituent as that of the compound H04 and a hydrocarbon central condensed ring, and is compared to the exemplified compound C02 of the present invention having a substituted 2′-m-terphenyl substituent and having a hydrocarbon central condensed ring, the comparative compound H04 had a lower T1 energy and a shallower estimated value of the LUMO level.

### Example 5

In this example, an organic light emitting device having a structure in which an anode, a hole transport layer, a light emitting layer, a hole blocking layer, an electron transport layer, and a cathode were sequentially provided on a substrate was formed by the following method.

A film having a thickness of 120 nm was formed on a glass substrate as an anode by a sputtering method using ITO and was used together with the substrate as a transparent conductive support substrate (ITO substrate). The following organic compound layers and electrode layers were sequentially vacuum-deposited on this ITO substrate by resistance heating in a vacuum chamber at a pressure of 10⁻⁵ Pa. At this stage, the electrodes were formed so that a facing area therebetween was 3 mm².

Hole transport layer (40 nm) HTL-1
Light emitting layer (30 nm)
Host material: Exemplified compound A04
Guest material: Ir-1 (10 wt %)
   Hole blocking layer (10 nm) HBL-1
   Electron transport layer (30 nm) ETL-1
   Metal electrode layer 1 (0.5 nm) LiF
   Metal electrode layer 2 (100 nm) Al

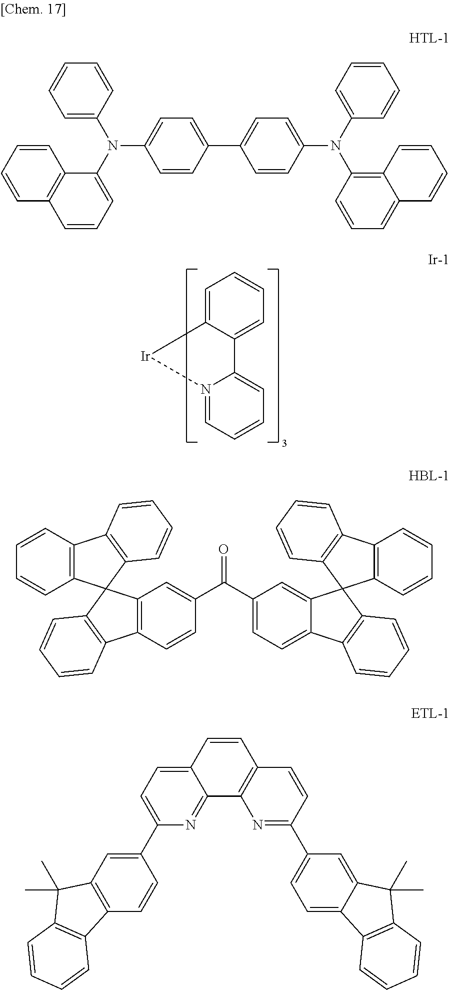

Next, The organic light emitting device was covered with a protective glass in a dry air atmosphere and was then sealed with an acrylic-based adhesive so as to prevent device degradation caused by moisture absorption. The organic light emitting device was obtained as described above.

When a voltage of 5.1 V was applied to the organic light emitting device thus obtained using the ITO electrode as a positive electrode and the Al electrode as a negative electrode, green light emission having a light emitting efficiency of 60.8 cd/A and a brightness of 2,000 cd/m² was observed. In addition, in this device, the CIE chromaticity coordinates were (x, y)=(0.30, 0.63).

### Example 6

A device was formed by a method similar to that of Example 5 except that in Example 5, the exemplified compound A06 was used as a host material of the light emitting layer instead of using the exemplified compound A04. In addition, the device thus obtained was evaluated in a manner similar to that of Example 5. The results are shown in Table 4.

### Example 7

A device was formed by a method similar to that of Example 5 except that in Example 5, the exemplified compound C02 was used as a host material of the light emitting layer instead of using the exemplified compound A04. In addition, the device thus obtained was evaluated in a manner similar to that of Example 5. The results are shown in Table 4.

### Comparative Example 2

A device was formed by a method similar to that of Example 5 except that in Example 5, the comparative compound H01 was used as a host material of the light emitting layer instead of using the exemplified compound A04. In addition, the device thus obtained was evaluated in a manner similar to that of Example 5. The results are shown in Table 4.

### Comparative Example 3

A device was formed by a method similar to that of Example 5 except that in Example 5, the comparative compound H03 was used as a host material of the light emitting layer instead of the exemplified compound A04. The device thus obtained was evaluated in a manner similar to that of Example 5. The results are shown in Table 4.

### Comparative Example 4

A device was formed in a manner similar to that of Example 5 except that in Example 5, the comparative compound H04 was used as a host material of the light emitting layer instead of using the exemplified compound A04. In addition, the device thus obtained was evaluated in a manner similar to that of Example 5. The results are shown in Table 4.

TABLE 4

| | LIGHT EMITTING | | APPLIED | LIGHT EMITTING |
|---|---|---|---|---|
| | LAYER | CIE | VOLTAGE | EFFICIENCY |
| | HOST MATERIAL | CHROMATICITY | @2000 cd/m² (V) | @2000 cd/m² (cd/A) |
| EXAMPLE 5 | EXEMPLIFIED | (0.30, 0.63) | 5.1 | 60.8 |
| | COMPOUND A04 | | | |
| EXAMPLE 6 | EXEMPLIFIED | (0.30, 0.64) | 5.0 | 63.7 |
| | COMPOUND A06 | | | |
| EXAMPLE 7 | EXEMPLIFIED | (0.30, 0.63) | 5.3 | 59.8 |
| | COMPOUND C02 | | | |
| COMPARATIVE | COMPARATIVE | (0.33, 0.63) | 6.2 | 47.7 |
| EXAMPLE 2 | COMPOUND H01 | | | |
| COMPARATIVE | COMPARATIVE | (0.33, 0.62) | 5.9 | 45.2 |
| EXAMPLE 3 | COMPOUND H03 | | | |
| COMPARATIVE | COMPARATIVE | (0.31, 0.63) | 6.8 | 39.3 |
| EXAMPLE 4 | COMPOUND H04 | | | |

### Example 8

In this example, in an organic light emitting device having a structure in which an anode, a hole transport layer, a light emitting layer, an electron transport layer, and a cathode were sequentially provided on a substrate, a device in which the hole transport layer was formed of two layers having different ionization potentials was formed by the following method.

On an ITO substrate formed by a method similar to that of Example 5, the following organic compound layers and electrode layers were sequentially vacuum-deposited by resistance heating at a pressure of 10⁻⁵ Pa in a vacuum chamber. At this stage, the electrodes were formed so that a facing electrode area therebetween was 3 mm².

Hole transport layer 1 (13 nm) HTL-1
Hole transport layer 2 (20 nm) HTL-2
Light emitting layer (40 nm)
Host material: Exemplified compound B01
Guest material: Ir-11 (10 wt %)
   Hole blocking layer (10 nm) HBL-1
   Electron transport layer (30 nm) ETL-2
   Metal electrode layer 1 (0.5 nm) LiF
   Metal electrode layer 2 (100 nm) Al

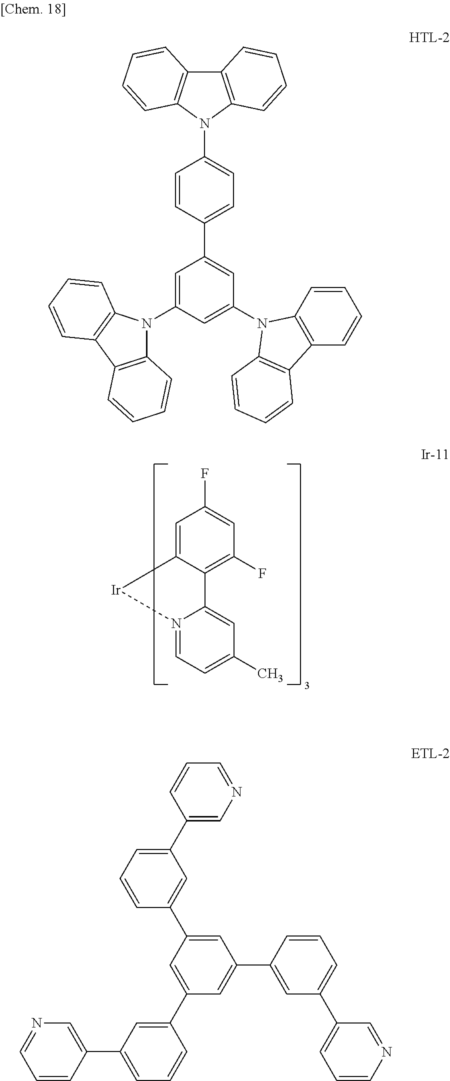

Next, the organic light emitting device was covered with a protective glass in a dry air atmosphere and was then sealed with an acrylic-based adhesive so as to prevent device degradation caused by moisture absorption. The organic light emitting device was obtained as described above.

When a voltage of 6.1 V was applied to the organic light emitting device thus obtained using the ITO electrode as a positive electrode and the Al electrode as a negative electrode, green light emission having a light emitting efficiency of 26.5 cd/A and a brightness of 2,000 cd/m² was observed. In addition, in this device, the CIE chromaticity coordinates were (x, y)=(0.16, 0.35).

### Comparative Example 5

A device was formed by a method similar to that of Example 8 except that in Example 8, the comparative compound H02 was used as a host material of the light emitting layer instead of using the exemplified compound B01. In addition, the device thus obtained was evaluated in a manner similar to that of Example 8. The results are shown in Table 5.

TABLE 5

| | LIGHT EMITTING | | APPLIED | LIGHT EMITTING |
|---|---|---|---|---|
| | LAYER | CIE | VOLTAGE | EFFICIENCY |
| | HOST MATERIAL | CHROMATICITY | @2000 cd/m² (V) | @2000 cd/m² (cd/A) |
| EXAMPLE 8 | EXEMPLIFIED | (0.16, 0.35) | 6.1 | 26.5 |
| | COMPOUND B01 | | | |
| COMPARATIVE | COMPARATIVE | (0.18, 0.38) | 7.9 | 14.0 |
| EXAMPLE 5 | COMPOUND H02 | | | |

As described above, the m-terphenyl compound according to the present invention is a novel compound having a high T1 energy and a deep LUMO level, and when this novel compound is used for an organic light emitting device, a light emitting device having a low driving voltage and a high light emitting efficiency can be obtained.

While the present invention has been described with reference to exemplary embodiment, it is to be understood that the invention is not limited to the disclosed exemplary embodiment. The scope of the following claims is to be accorded the broadest interpretation so as to encompass all such modifications and equivalent structures and functions.

This application claims the benefit of Japanese Patent Application No. 2010-099153, filed Apr. 22, 2010, which is hereby incorporated by reference herein in its entirety.

1. A m-terphenyl compound represented by the following general formula [1] 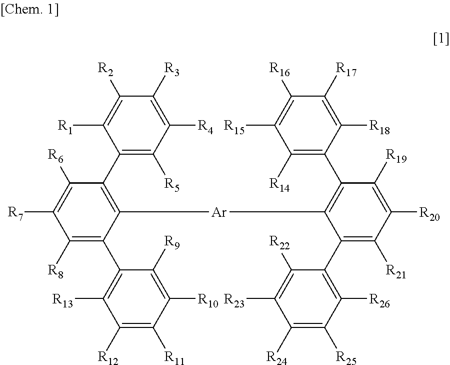
wherein in formula [1], R₁ to R₂₆ each independently indicate a hydrogen atom,
Ar is selected from arylene groups represented by formula [2], and 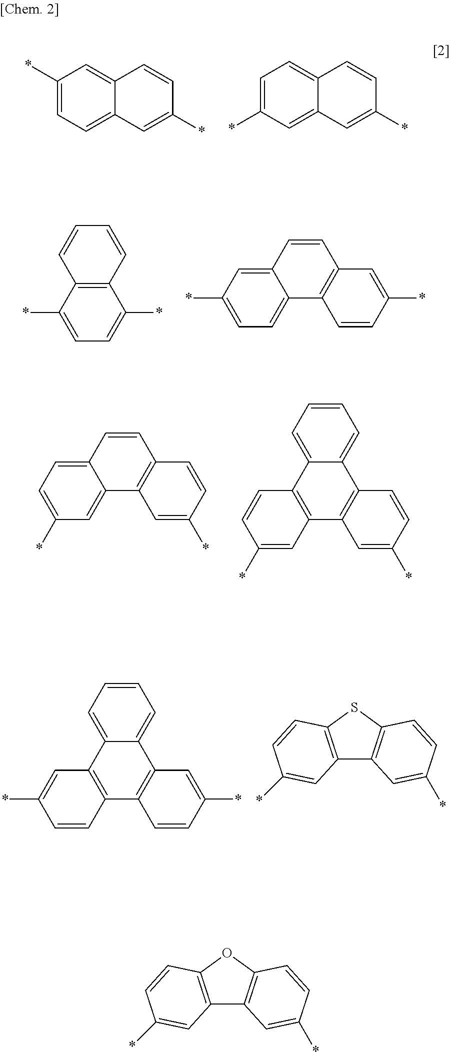
in formula [2], * indicates a bonding site to a m-terphenyl group.

2. (canceled)

3. An organic light emitting device comprising:
a pair of electrodes, and
an organic compound layer provided therebetween,
wherein the organic compound layer includes the m-terphenyl compound according to claim 1.

4. The organic light emitting device according to claim 3,
wherein the organic compound layer is a light emitting layer.

5. The organic light emitting device according to claim 4,
wherein the light emitting layer includes a host material and a guest material, and
the host material contains the m-terphenyl compound.

6. The organic light emitting device according to claim 5,
wherein the guest material is a phosphorescence material.

7. The organic light emitting device according to claim 6,
wherein the phosphorescence material contains an iridium complex.

8. An image display device comprising:
the organic light emitting device according to claim 3;
and
a switching element connected thereto.

9. An image display device comprising:
the organic light emitting device according to claim 4;
and
a switching element connected thereto.

10. An image display device comprising:
the organic light emitting device according to claim 5;
and
a switching element connected thereto.

11. An image display device comprising:
the organic light emitting device according to claim 6;
and
a switching element connected thereto.

12. An image display device comprising:
the organic light emitting device according to claim 7;
and
a switching element connected thereto.
